(19) United States
(12) Patent Application Publication
(10) Pub. No.: US 2006/0116392 A1
(43) Pub. Date: Jun. 1, 2006

(54) Methods for treating anxiety disorders and related article of manufacture

(76) Inventors: Rene Hen, Tenafly, NJ (US); Luca Santarelli, San Francisco, CA (US); Cristina Alberini, New York, NY (US); Maria Milekie, New York, NY (US)

Correspondence Address: COOPER & DUNHAM, LLP, 1185 AVENUE OF THE AMERICAS, NEW YORK, NY, 10036, US

(21) Appl. No.: 11/144,421

(22) Filed: Jun. 3, 2005

Related U.S. Application Data
(60) Provisional application No. 60/577,121, filed on Jun. 3, 2004.

(51) Int. Cl. A61K 31/4745

(52) U.S. Cl. 514/291

(57) ABSTRACT

This invention provides a method for treating a subject afflicted with an anxiety disorder whose symptoms are exacerbated by the subject's exposure to a predetermined eliciting event, which method comprises administering to the subject a therapeutically effective amount of a protein synthesis inhibitor before, during the time, and/or after the subject intentionally recalls the eliciting event. Furthermore, this invention provides a method for inhibiting in a subject the onset of an anxiety disorder in response to an anxiety disorder-causing event. Finally, this invention provides a related article of manufacture.

This application claims the benefit of U.S. Provisional Application No. 60/577,121, filed Jun. 3, 2004, the contents of which are hereby incorporated by reference.

This invention was made with support under United States Government Grant No. ______. Accordingly, the United States Government has certain rights in this invention.

Throughout this application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

In human addicts, the exposure to reminder cues evokes a strong increase in drug-seeking behavior. This compulsive addiction response has been hypothesized to rely on memory-like consolidation mechanisms resulting from a gradual strengthening of the behavior, or consolidation, induced by the reinforcing/rewarding action of the drug. Thus, disrupting this consolidation may weaken drug-seeking behavior. The consolidation of new memories and the preservation of consolidated memories after recall depend upon a transient phase of protein biosynthesis.

In human addicts, exposure to environmental stimuli previously associated with a drug has been shown to increase drug-seeking behavior (Ludwig and Stark, 1974; O'Brien et al., 1977). This conditioned response is long lasting and can occur despite years of abstinence from drug use (O'Brien et al., 1992).

Animal models used to study how a neutral context becomes associated with rewarding properties of drugs use a procedure known as conditioned place preference (“CPP”). During CPP, the animal develops a preference for a context repeatedly associated with a drug (Beach, 1957; Carr et al., 1989). Using this and other behavioral tasks based on exposure to cues that elicit craving, it has been observed that addiction and memory have several common features (reviewed in Wickelgren, 1998; Hyman and Malenka, 2001; Nesler, 2002). First, drugs of abuse and reinforcers in general can be viewed as memory modulators or enhancers because their effects are part of the learning experience (reviewed in Landauer, 1969; White, 1996). Second, many compounds that impair memory (Li et al., 1997; Zhou et al., 1999; London et al., 1995) or inhibit memory-associated molecular pathways (Fan et al., 1999; Lu et al., 2000) attenuate drug tolerance and dependence. Finally, brain structures essential for memory formation, including hippocampus and amygdala, also mediate the addictive response. Lesions of the amygdala impair CPP (Everitt et al., 1991; McDonald and White, 1993) and electrical stimulation of the hippocampus elicits cocaine-seeking behavior (Vorel et al., 2001).

A basic and essential feature of the consolidation of a new memory is its requirement for gene expression (Davis and Squire, 1984). Indeed, the disruption of protein synthesis after a learning event, during a phase required to strengthen and stabilize the memory (consolidation phase), prevents the formation of that memory. Once consolidated, the memory is stable and insensitive to the disruptive effects of protein synthesis inhibitors (Squire and Alvarez, 1995). However, old, consolidated memories, if retrieved, again become labile and require protein synthesis to be preserved (Judge and Quartermain, 1982; Nader et al., 2000; Taubenfeld et al., 2001; Milekic and Alberini, 2002; Anokhin et al., 2002; Kida et al., 2002; Sara, 2000; Debiec et al., 2002).

### SUMMARY OF THE INVENTION

This invention provides a method for treating a subject afflicted with an anxiety disorder whose symptoms are exacerbated by the subject's exposure to a predetermined eliciting event, which method comprises administering to the subject a therapeutically effective amount of a protein synthesis inhibitor before, during the time, and/or after the subject intentionally recalls the eliciting event.

This invention further provides a method for inhibiting in a subject the onset of an anxiety disorder in response to an anxiety disorder-causing event, wherein the disorder would be characterized by symptoms which would be exacerbated by the subject's exposure to a predetermined eliciting event, which method comprises administering to the subject a prophylactically effective amount of a protein synthesis inhibitor before, during the time and/or after the subject intentionally recalls the eliciting event.

This invention further provides an article of manufacture comprising (a) a packaging material having therein a protein synthesis inhibitor, and (b) a label indicating a use for the inhibitor in treating, or inhibiting the onset of, an anxiety disorder in a subject.

### BRIEF DESCRIPTION OF THE FIGURES

FIGS. 1A-1B: Effect of protein synthesis inhibitors on the induction of morphine CPP

Values are expressed as mean±SEM time spent in the drug associated-chamber during pre-conditioning and the test phases. *p<0.05, **p<0.01

(1A) Groups of rats were conditioned for 4 days to either vehicle (Ve only, n=8, white bars) or morphine (Morp, n=15). At the end, the morphine-conditioned rats were either injected s.c. with vehicle (Morp/Ve, n=8, black bar) or inhibitor (Morp/Inhibitor n=7 of which n=4 with cycloheximide and n=3 with anisomycin, striped bars). All rats were tested 24 hr and 1 week later (post-conditioning).

(1B) Rats received daily administration of cycloheximide (Cy, n=8, black bars), or vehicle (Ve, n=8, white bars) immediately after each conditioning session. CPP was tested 24 hr, 1 and 4 weeks later.

FIG. 2: Protein synthesis is not required for the maintenance of morphine CPP after contextual reactivation

Rats were conditioned to morphine and tested after 1 week (Test 1) to reactivate the CPP. Immediately after Test 1, half of the rats were injected with cycloheximide or anisomycin (Inhibitor n=8 of which n=4 with cycloheximide and n=4 with anisomycin, black bar) and the other half with vehicle (Ve n=7, white bar). When re-tested 24 hr later (Test 2), all rats maintained a similar CPP score. Values are expressed as mean±SEM time spent in the drug associated-chamber during pre-conditioning and the test phases.

FIGS. 3A-3D: A consolidated CPP is disrupted by protein synthesis inhibitors administered after a single reconditioning to the drug

Values are expressed as mean±SEM time spent in the drug associated-chamber during pre-conditioning and the test phases. *p<0.05, **p<0.01, ***p<0.001

(3A) Rats were conditioned to morphine and one week later received one more conditioning session (reconditioning). Immediately after, half of the animals were injected with cycloheximide or anisomycin (Inhibitors n=10 of which n=6 with cycloheximide and n=4 with anisomycin, black bar) and the other half with vehicle (Ve, n=8, white bar). Rats were tested 24 hr and 1 week later (post-reconditioning).

(3B) Animals were conditioned and reconditioned the same way as in (3A), but received two injections of cycloheximide, 5 hr apart (Cy, n=10, except for the 4 week test n=7, black bar) or vehicle (Ve, n=11, white bar) immediately after reconditioning. Rats were tested 24 hr, 1, 2 and 4 weeks later.

(3C) The conditioning context was omitted and rats (n=8) were injected in their home cage with morphine and 30 min later with cycloheximide at the same time point as in (3A) and (3B). CPP was tested 24 hr after injection (post-injection).

(3D) Rats were injected only with cycloheximide (n=9) in the home cage at the same time points as in (3A) and (3B) without receiving either morphine or exposure to the conditioning context. CPP was tested 24 hr after the last injection (post-injection).

### DETAILED DESCRIPTION OF THE INVENTION

Definitions

As used in this application, except as otherwise expressly provided herein, each of the following terms shall have the meaning set forth below.

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. “Administering” can also be performed, for example, once, a plurality of times, and/or over one or more extended periods.

As used herein, “agents” include, but are not limited to, small molecules, proteins, nucleic acids, carbohydrates, lipids and any other molecules or compounds.

As used herein, “anxiety disorder” shall mean all disorders as defined by the diagnostic criteria contained or included in the Diagnostic and Statistical Manual of Mental Disorders 4^{th} Edition (DSM-IV).

As used herein, “conditioned place preference”, “place preference” and “CPP” are synonymous, and shall mean a behavioral task that is used to detect the addictive or appetitive properties of a substance or an experience.

As used herein, “inhibiting” the onset of a disorder shall mean either lessening the likelihood of the disorder's onset, or preventing the onset of the disorder entirely. In the preferred embodiment, inhibiting the onset of a disorder means preventing its onset entirely.

As used herein, “pharmaceutically acceptable carriers” include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

As used herein, “protein synthesis inhibitor” shall mean any agent which, when introduced to or into a cell, inhibits some or all of the protein synthesis in the cell which, in the agent's absence, would otherwise occur.

As used herein, “subject” shall mean any animal, such as a primate, mouse, rat, guinea pig or rabbit. In the preferred embodiment, the subject is a human.

As used herein, “treating” a subject afflicted with a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

### EMBODIMENTS OF THE INVENTION

This invention provides a method for treating a subject afflicted with an anxiety disorder whose symptoms are exacerbated by the subject's exposure to a predetermined eliciting event, which method comprises administering to the subject a therapeutically effective amount of a protein synthesis inhibitor before, during the time, and/or after the subject intentionally recalls the eliciting event.

In one embodiment of the instant method, the subject is human. In another embodiment, the subject is a rat.

Anxiety disorders treated by this method include, for example, drug addiction, post-traumatic stress disorder, phobia, obsessive compulsive disorder, and an anxiety disorder characterized by panic attacks.

Protein synthesis inhibitors which can be used in this method include, without limitation, rapamycin, CCI-779, and RAD-001, and any combination thereof.

Therapeutically effective amounts of protein synthesis inhibitors for humans can be determined based on animal data and routine computational methods. In one embodiment, the subject is human, the protein synthesis inhibitor is rapamycin, and the therapeutically effective amount per subject is selected from between 1 mg and 2400 g; between 300 mg and 2100 mg; between 600 mg and 1800 mg; between 900 mg and 1500 mg; between 1100 mg and 1400 mg; and between 1200 mg and 1300 mg.

Eliciting events (i.e. events which can trigger a conditioned place preference in a subject) include, for example, visual, olfactory, tactile and gustatory stimuli. An eliciting event can be encountered by a subject physically or in the subject's imagination. The following are examples of stimuli which could trigger conditioned place preference in a subject: visual (e.g. viewing a photograph of a location where the subject's drug addiction occurred); olfactory (e.g. smelling the drug or drug paraphernalia associated with the subject's addiction); tactile (e.g. touching, manually and or orally, the drug or drug paraphernalia associated with the subject's addiction); and or gustatory (e.g. drinking beverage and/or eating food consumed at or near the time when drug use or addiction occurred).

In the instant method, the protein synthesis inhibitor can be administered before, during the time, and/or after the subject intentionally recalls the eliciting event. Specifically, the inhibitor can be administered (i) before; (ii) during the time; (iii) after; (iv) before and during the time; (v) during the time and after; (vi) before and after; or (vii) before, during the time and after the subject recalls the eliciting event.

In one embodiment, the protein synthesis inhibitor is administered 2 hours, 1 hour, 30 minutes, or 10 minutes before the eliciting event. In still a further embodiment, the protein synthesis inhibitor is administered 2 hours, 1 hour, 30 minutes, or 10 minutes after the eliciting event.

In the preferred embodiment, the protein synthesis inhibitor, when administered, is admixed with a pharmaceutically acceptable carrier.

This invention further provides a method for inhibiting in a subject the onset of an anxiety disorder in response to an anxiety disorder-causing event, wherein the disorder would be characterized by symptoms which would be exacerbated by the subject's exposure to a predetermined eliciting event, which method comprises administering to the subject a prophylactically effective amount of a protein synthesis inhibitor before, during the time and/or after the subject intentionally recalls the eliciting event.

In an embodiment of the instant method, the anxiety disorder is post-traumatic stress disorder caused by experiencing (e.g. witnessing and/or being subjected to) an act of violence. Acts of violence include, for example, sexual and non-sexual assaults.

This invention further provides an article of manufacture comprising (a) a packaging material having therein a protein synthesis inhibitor, and (b) a label indicating a use for the inhibitor in treating, or inhibiting the onset of, an anxiety disorder in a subject.

Wherever applicable, the various embodiments of the instant therapeutic method are envisioned, mutatis mutandis, with respect to the instant prophylactic method and article of manufacture.

This invention is illustrated in the Experimental Details section which follows. This section is set forth to aid in an understanding of the invention but is not intended to, and should not be construed to, limit in any way the invention as set forth in the claims which follow thereafter.

Experimental Details

Results

The Induction of Morphine CPP Requires Protein Synthesis

The first set of experiments determined whether the induction of morphine CPP requires protein synthesis. Rats were conditioned to morphine or vehicle and at the end of the four-day conditioning session, half of the morphine-conditioned rats received a single subcutaneous (s.c.) dose of the protein synthesis inhibitor anisomycin or cycloheximide and the other half was injected with vehicle solution. CPP was tested after 24 hours. To determine whether the effect was stable, the animals were retested 1 week later.

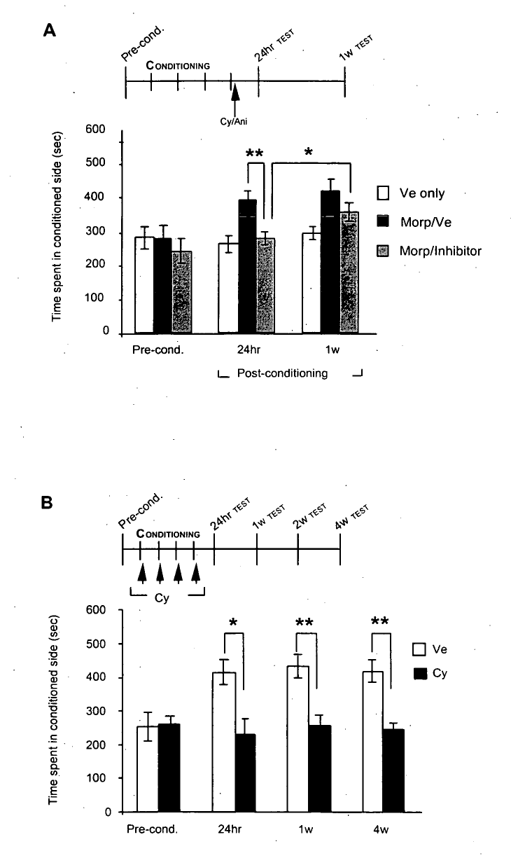

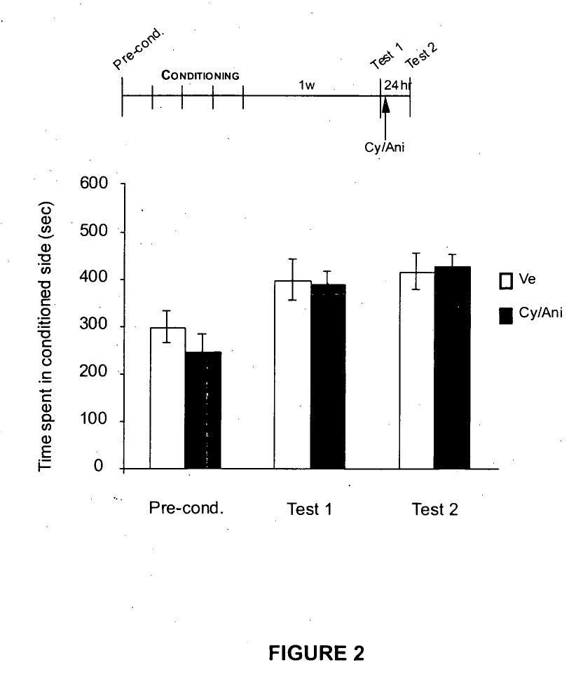

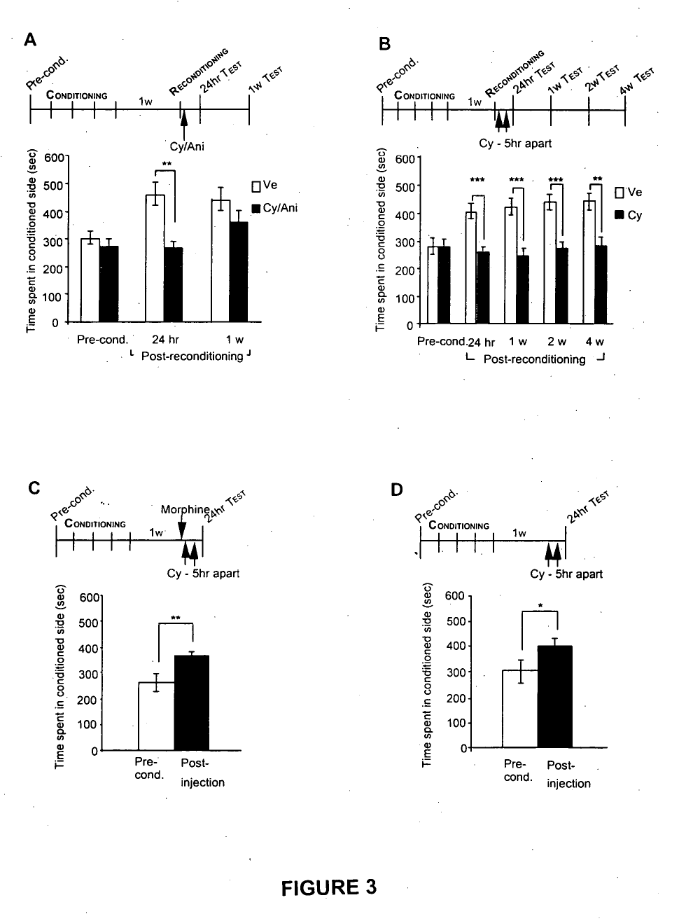

At 24 hours, both anisomycin and cycloheximide completely blocked morphine CPP (FIG. 1A). A one-way analysis of variance (ANOVA) that compared the CPP scores across vehicle, morphine and morphine-with-inhibitor-injected groups revealed that there was a significant group effect (F_{(2, 22)}=7.6, p<0.01). A Newman-Keul post-hoc test showed that the animals treated with anisomycin or cycloheximide had a significantly lower conditioning score (280.1±17.5 sec; p<0.01) compared to morphine-conditioned-vehicle injected controls (390.3±31.5 sec). Moreover, their post-conditioning score was not significantly different from that of the vehicle-conditioned animals (262.0±73.8 sec) or their pre-conditioning score (243.3±36.5 sec). Because both anisomycin and cycloheximide produced a similar effect, the CPP score of the two groups were combined. However, when the same animals were re-tested 1 week later they showed a partial, but significant recovery (p<0.05 t test) of the place preference (358.9±28.0 sec) compared to their 24 hour conditioning score (FIG. 1A). This suggested that inhibition of protein synthesis at the end of a four-day conditioning impaired CPP only transiently. One possible explanation for this recovery is that a single dose of inhibitor at the end of a four-day conditioning does not sufficiently block the protein synthesis required for CPP induction (Flood et al., 1975) and, instead, just delays the conditioning process. Further experimentation was needed to determine whether a more extended inhibition of protein synthesis was able to persistently block CPP. Groups of rats were treated as above but received two injections of cycloheximide, one immediately after the last conditioning session and the second 5 hours later. In parallel, the rates of protein synthesis inhibition, over time, following a single or double injection were established. A single injection produced a 70% inhibition of protein synthesis at 1 hour that decreased to 23% at 6 hours after injection. With a double injection, 5 hours apart, the rate of protein synthesis inhibition at 6 hours was maintained at 71%. The results after two injections were similar to those after a single injection; in fact, the CPP of rats that received two injection of cycloheximide was inhibited at 24 hours, but recovered a week later (data not shown).

Experimentation was needed to test the possibility that blocking protein synthesis after each daily conditioning produced a stable loss of CPP. Groups of rats received an injection of either cycloheximide or vehicle every day at the end of each conditioning trial and were then tested 24 hours, 1 week and 4 weeks later. A student t test that compared the CPP score between morphine-conditioned vehicle-injected and morphine-conditioned cycloheximide-injected groups revealed that, at 24 hours after conditioning, the latter showed a complete inhibition of CPP compared to the former (226.6±47.2 sec and 410.9±36.3 sec, respectively, p<0.05). This inhibition persisted at 1 week (254.8±32.8 sec and 429.4±33.1 sec, respectively, p<0.01) and 4 weeks (243.3±21.8 sec and 416.8±31.4 sec, respectively, p<0.01) post-conditioning (FIG. 1B). Thus, blocking protein synthesis after each conditioning session persistently inhibits CPP.

Reconditioning, but Not Contextual Recall, Returns CPP to a Labile State.

The purpose of second set of experiments was to determine whether morphine CPP becomes labile if recalled. Groups of rats were conditioned to morphine for 4 days. One week after the end of conditioning, the rats were tested for place preference and, as expected, showed a strong preference for the context associated with morphine (FIG. 2). This test recalled the experience (Test 1), or, in other words, reactivated CPP. Immediately after Test 1, half of the rats received an injection of cycloheximide or anisomycin and half were injected with vehicle. The animals were re-tested 24 hours later (Test 2). No change in their CPP score was observed and all rats showed a strong preference for the conditioned side. (Test1-vehicle: 394.4±42.6 sec; Test1-inhibitor: 387.8±25.8 sec; Test2-vehicle 413.3±35.9 sec; Test2-inhibitor 425.9±25.3 sec) (FIG. 2). Because both cycloheximide and anisomycin-treated rats performed similarly, their scores were combined. Therefore, it appears that, unlike fear memories (Judge and Quartermain, 1982; Nader et al., 2000; Milekic and Alberini, 2002; Anokhin et al. 2002), CPP does not become sensitive to protein synthesis inhibitors after contextual recall.

However, it is important to consider that during morphine CPP, the drug may serve as an internal reinforcer and the animal experiences a strong reward. It remained unknown whether CPP may become labile if the rewarding state of the drug experience is concomitantly reactivated with the contextual cues (reconditioning). Therefore, it was examined whether a previously formed CPP becomes labile after a reconditioning session. Rats were conditioned to morphine for 4 days and, 1 week later, were exposed to another, single conditioning session. Immediately after this session, half of the animals received an injection of inhibitor (cycloheximide or anisomycin) and the other half of vehicle solution and were then all retested 24 hours and 1 week later. A student t test that compared the CPP scores between the vehicle-injected and inhibitor-injected groups revealed that the inhibitor caused a significant disruption of CPP at 24 hours after reconditioning (inhibitor: 261.5±33.9 sec, vehicle: 461.6±42.5 sec, p<0.01), but this disruption did not persist 1 week later (inhibitor: 365.4±45.1 sec, vehicle 442.3±29.1 sec) (FIG. 3A).

It was then tested whether blocking protein synthesis after reconditioning for a longer time would result in a stable disruption of CPP. Groups of rats were treated as above but received two injections of cycloheximide, one immediately after reconditioning and the second 5 hours later, a protocol that, as described in the first set of experiments, block 70% of protein synthesis for several hours.

A double injection of cycloheximide after reconditioning completely and persistently blocked CPP (FIG. 3B). The rats injected with cycloheximide or vehicle were repeatedly tested 24 hours (cyclo: 264.1±13.3 sec, vehicle: 396.5±26.7 sec), 1 week (cyclo: 239.9±34.4 sec, vehicle: 416.8±26.8 sec), 2 weeks (cyclo: 270.1±27.2 sec, vehicle: 432.2±27.6 sec) and 4 weeks after reconditioning (cyclo: 278.4±32.5 sec, vehicle: 432.5±29.6 sec), and, cycloheximide-injected rats never recovered the preference for the conditioned compartment. A student t test revealed that cycloheximide compared to vehicle treatment significantly and persistently disrupted CPP (p<0.001 for 24 hours, 1 week, 2 weeks and p<0.01 for 4 weeks) and that at all timepoints of testing the CPP scores of cycloheximide-treated rats were similar to their pre-conditioning score (273.7±30.6 sec).

To investigate whether the blocking effect of the inhibitors on CPP was context-specific, the conditioning context was omitted, that is morphine and cycloheximide were administered in the homecages at the same times after conditioning. No blocking effect was observed and the rats showed a significant CPP (post-conditioning: 362.22±17.4 sec; pre-conditioning: 260.9±32.9 sec, p<0.05) (FIG. 3C). Similarly, if morphine was also omitted and the rats received only the inhibitor in their homecage at the same time after conditioning, they showed significant CPP; indeed, their post-conditioning score was significantly higher than (399.8±31.6 sec, p<0.01) their preconditioning score (295.9±44.1) (FIG. 3D), suggesting that, once consolidated, CPP does not become labile following the re-experience of the drug in a novel context.

Discussion

The results show that the induction of morphine CPP is disrupted by the inhibition of protein synthesis during conditioning. Moreover, they show that a previously established CPP returns to a labile state if reactivated by a single reconditioning session and requires protein synthesis in order to be maintained.

Hence, it appears that the requirements for protein synthesis underlying induction and maintenance of CPP are, to a certain extent, similar to those underlying the consolidation and the post-reactivation maintenance of memory, respectively (Davis and Squire, 1984; Nader et al., 2000). However, the requirement for protein synthesis after memory or CPP reactivation has distinctive features. The temporal lobe-dependent memories, including fear-conditioning, spatial and object recognition memories (Nader et al., 2000; Taubenfeld et al., 2001; Milekic and Alberini, 2002; Anokhin et al., 2002, Nader et al., 2002; Kida et al., 2002, Debiec et al., 2002; Przybyslawski and Sara, 1997; Przybyslawski et al., 1999; Kelly et al., 2003) return to a labile state following the re-experience of the conditioned stimulus only. In contrast, the de-stabilization of CPP requires that both the effect of the drug and the conditioned context be concomitantly re-experienced. Indeed, although they may induce molecular changes (Thomas et al., 2003), the re-exposure to the conditioned context alone or the re-experience of the drug in a new context are not sufficient for destabilizing CPP. This indicates that a CPP representation that has been consolidated, and therefore needs to be reactivated in order to become labile, involves the associations between the internal state induced by the drug and the representation of a specific context. The neural structures mediating the consolidation and reactivation of this CPP representation and the nature of the proteins required for these processes remains to be determined.

The disruption of the morphine conditioning by the action of protein synthesis inhibitors is stable for several weeks and no recovery was observed at any time in the experiments. This suggests that the synthesis of proteins during conditioning or immediately after reconditioning is a limiting step for CPP induction and maintenance.

Addiction is a complex process involving interactions between cognitive and autonomic responses. These studies have demonstrated the requirement for protein synthesis for at least one behavioral manifestation of addiction, the contextually-conditioned response. It is possible that the effect of protein synthesis inhibitors described here is specific for memory-related/associative mechanisms of addiction. The determination of whether and how the requirement for protein synthesis underlies other aspects of addiction including withdrawal, sensitization and tolerance will address this question. Previous reports have shown that the induction of psychostimulant sensitization requires the synthesis of new proteins (Sorg and Ulibarri, 1995; Karler et al., 1993) and that following contextual reactivation, protein synthesis inhibitors do not affect sensitization but disrupt amphetamine-conditioned locomotion (Thomas and Robinson. Reconsolidation of amphetamine-conditioned locomotion but not psychomotor sensitization. Abstract at 32^{nd} Society for Neuroscience Meeting, Orlando, 2002).

The results indicate that a temporally-restricted combination of both behavioral and pharmacological intervention that target newly synthesized proteins can be used to weaken behavioral responses to drug addiction.

Experimental Procedures

Conditioned Place Preference (CPP)

Adult Long Evans rats (200-250 grams) were used for all the experiments. On day 1, the rats received a single pre-exposure test in a box composed of two distinct compartments (Pre-conditioning). The time spent in each chamber over 10 minutes was recorded (unconditioned preference) and then the animals were returned to their homecages. Animals showing a strong unconditioned preference (>540 s) were discarded. On the subsequent 4 days, place preference conditioning was conducted using an unbiased procedure. In each experimental group, the animals received a subcutaneous injection of either morphine (10 mg/kg) or saline (vehicle solution). Half of both groups were confined for 30 minutes in the spontaneously preferred compartment and the other half in the non-preferred compartment. Testing consisted of allowing the animals free access to both chambers for 10 minutes and recording the amount of time that the animals spend in the conditioned chamber (CPP score). All protocols complied with the N.I.H. Guide for the Care and Use of Laboratory Animals and were approved by the Mount Sinai School of Medicine Animal care Committees.

Protein Synthesis Inhibitors

Anisomycin and Cycloheximide were injected s.c. at 210 mg/Kg (Milekic and Alberini, 2002) and 2.8 mg/Kg (Squire et al., 1980), respectively. Anisomycin was dissolved as previously described (Milekic and Alberini, 2002). Cycloheximide was dissolved in DMSO and finally diluted in 1% DMSO/saline. Vehicle solutions were prepared accordingly.

Inhibition of Protein Synthesis

Inhibition of protein synthesis was assessed according to Rosenblum et al. (1993) with modifications. Rats were injected with 125 μCi L-[³⁵S]methionine (i.p.) and immediately after received an injection of either protein synthesis inhibitors, (cycloheximide or anisomycin) or vehicle solution. The rats were sacrificed one hour after the last inhibitor injection. The brains were homogenized in 5 volumes of cold lysis buffer (1% SDS, 60 mM Tris.Cl pH 7.4, 62.4 mM imidazole, 40 mM DTT, 10% glycerol). The homogenates were boiled for 5 min and centrifuged at 18,000 g for 20 min at room temperature. Equal amount of proteins were precipitated with 20% TCA and the radioactivity was counted. The inhibition of protein synthesis was calculated as: [1−(CPM_{inhibitor}/CPM_{vehicle})]×100.

### REFERENCES

Anokhin, K. V., Tiunova, A. A., and Rose, S. P. (2002). Reminder effects—reconsolidation or retrieval deficit? Pharmacological dissection with protein synthesis inhibitors following reminder for a passive-avoidance task in young chicks. Eur. J. Neurosci. 15, 1759-1765.
Beach, H. D. (1957). Morphine addiction in rats. Can. J. Psychology 11, 104-112.
Carr, G. D., Fibiger, H. C., and Phillips, A. G. (1989). Conditioned place preference as a measure of drug reward. In: The Neuropharmacological Basis of Reward. Liebman, J. M., and Cooper, S. J., eds., (Oxford, Clarendon Press), pp. 264-319.
Davis, H. P., and Squire, L. R. (1984). Protein synthesis and memory: a review. Psychol. Bull. 96, 518-559.
Debiec, J., LeDoux, J. E., and Nader, K. (2002). Cellular and systems reconsolidation in the hippocampus. Neuron. 36, 527-38.
Everitt, B. J., Morris, K. A., O'Brien, A., and Robbins, T. W. (1991). The basolateral amygdala-ventral striatal system and conditioned place preference: further evidence of limbic-striatal interactions underlying reward-related processes. Neuroscience 42, 1-18.
Fan, G. H., Wang, L. Z., Qiu, H. C., Ma, L., and Pei, G. (1999). Inhibition of calcium/calmodulin-dependent protein kinase II in rat hippocampus attenuates morphine tolerance and dependence. Mol. Pharmacol. 56, 39-45.
Flood, J. F. B., Bennet, E. L., Orme, A. E., and Rosensweig, M. R. (1975). Relation of memory formation to controlled amounts of brain protein synthesis. Physiol. Behav. 15, 97-102.
Hyman, S. E., and Malenka, R. C. (2001). Addiction and the brain: the neurobiology of compulsion and its persistence. Nat. Rev. Neurosci. 2, 695-703.
Judge, M. E., and Quartermain, D. (1982). Characteristics of retrograde amnesia following reactivation of memory in mice. Physiol. Behav. 28, 585-590.
Karler, R., Finnegan, K. T., and Calder, L. D. (1993). Blockade of behavioral sensitization to cocaine and amphetamine by inhibitors of protein synthesis. Brain Res. 603, 19-24
Kelly, A., Laroche, S., and Davis, S. (2003). Activation of mitogen-activated protein kinase/extracellular signal-regulated kinase in hippocampal circuitry is required for consolidation and reconsolidation of recognition memory. J. Neurosci. 23, 5354-5360.
Kida, S., Josselyn, S. A., de Ortiz, S. P., Kogan, J. H., Chevere, I. et al. (2002). CREB required for the stability of new and reactivated fear memories. Nat. Neurosci. 5, 348-355.
Kleim, J. A. B., Bruneau, R., Calder, K., Pocock, D., VandenBerg, P. M. et al. (2003). Functional organization of adult motor cortex is dependent upon continued protein synthesis. Neuron 40, 167-176.
Li, H. B., Matsumoto, K., Yamamoto, M., and Watanabe, H. (1997). NMDA but not AMPA receptor antagonists impair the delay-interposed radial maze performance of rats. Pharmacol. Biochem. Behav. 58, 249-253.
Landauer, T. K. (1969). Reinforcement as consolidation. Psych. Rev.76, 82-96
London, E. D., Kimes, A. S., and Vaupel, D. B. (1995). Inhibitors of nitric oxide synthase and the opioid withdrawal syndrome. NIDA Res. Monogr. 147, 170-181
Lu, L., Zeng, S., Liu, D., and Ceng, X. (2000). Inhibition of the amygdala and hippocampal calcium/calmodulin-dependent protein kinase II attenuates the dependence and relapse to morphine differently in rats. Neurosci. Lett. 291, 191-195.
Ludwig, A. M., and Stark, L. H. (1974). Alcohol craving. Subjective and situational aspects. Q. J. Stud. Alcohol 35, 899-905.
McDonald, R. J., and White, N. M. (1993). A triple dissociation of memory systems: hippocampus, amygdala, and dorsal striatum. Behav. Neurosci. 107, 3-22.
Milekic, M. H., and Alberini, C. M. (2002). Temporally graded requirement for protein synthesis following memory reactivation. Neuron 36, 521-525.
Nader, K., Schafe, G. E., and LeDoux, J. E. (2000). Fear memories require protein synthesis in the amygdala for reconsolidation after retrieval. Nature 406, 722-726.
Nestler, E. J. (2002). Common molecular and cellular substrates of addiction and memory. Neurobiol. Learn. Mem. 78, 637-647.
O'Brien, C. P., Testa, T., O'Brien, T. J., Brady, J. P., and Wells, B. (1977). Conditioned narcotic withdrawal in humans. Science 195, 1000-1002.
O'Brien, C. P., Childress, A. R., McLellan, A. T., and Ehrman, R. (1992). Classical conditioning in drug-dependent humans. Ann. N Y Acad. Sci. 654, 400-415.
Przybyslawski, J., and Sara, S. J. (1997). Reconsolidation of memory after its reactivation. Behav. Brain Res. 84, 241-246.
Przybyslawski, J., Roullet, P., and Sara, S. J. (1999). Attenuation of emotional and nonemotional memories after their reactivation: role of beta adrenergic receptors. J. Neurosci. 19, 6623-6628.
Robbins, T. W., and Everitt, B. J. (2002). Limbic-striatal memory systems and drug addiction. Neurobio.l Learn. Mem. 78, 625-636.
Rosenblum, K., Meiri, N., and Dudai, Y. (1993). Taste memory: the role of protein synthesis in gustatory cortex. Behav. Neural Biol. 59, 49-56.
Sara, S. J. (2000). Retrieval and reconsolidation: toward a neurobiology of remembering. Learn. Mem. 7, 73-84.
Sorg, B. A., and Ulibarri, C. (1995). Application of a protein synthesis inhibitor into the ventral tegmental area, but not the nucleus accumbens, prevents behavioral sensitization to cocaine. Synapse 20, 217-224.
Squire, L. R., Davis, H. P., Spanis, C. W., Gold, P. E., and Sternberg, D. B. Neurobiology of amnesia. Science 209, 836 (1980).
Squire, L. R., Alvarez, P. (1995). Retrograde amnesia and memory consolidation: a neurobiological perspective. Curr. Opin. Neurobiol. 5, 169-177
Taubenfeld, S. M., Milekic, M. H., Monti, B., and Alberini, C. M. (2001) The consolidation of new but not reactivated memory requires hippocampal C/EBPbeta. Nat. Neurosci. 4, 813-818.
Thomas, K. L., Arroyo, M., and Everitt, B. J. (2003). Induction of the learning and plasticity-associated gene Zif268 following exposure to a discrete cocaine-associated stimulus. Eur. J. Neurosci. 17, 1964-1972.
Vorel, S. R., Liu, X., Hayes, R. J., Spector, J. A., and Gardner, E. L. (2001). Relapse to cocaine-seeking after hippocampal theta burst stimulation. Science 292, 1175-1178.
White, N. M. (1996). Addictive drugs as reinforcers: multiple partial actions on memory systems. Addiction 91, 921-965.
Wickelgren, I. (1998). Teaching the brain to take drugs. Science 280, 2045-2046.
Zhou, H., Ge, X., Wang, L. Z., Ma, L., and Pei, G. (1999). Attenuation of morphine tolerance and dependence in scopolamine-treated rats. Neuroreport 10, 2007-2010.

What is claimed is:

1. A method for treating a subject afflicted with an anxiety disorder whose symptoms are exacerbated by the subject's exposure to a predetermined eliciting event, which method comprises administering to the subject a therapeutically effective amount of a protein synthesis inhibitor before, during the time, and/or after the subject intentionally recalls the eliciting event.

2. The method of claim 1, wherein the subject is human.

3. The method of claim 1, wherein the anxiety disorder is drug addiction.

4. The method of claim 1, wherein the anxiety disorder is post-traumatic stress disorder.

5. The method of claim 1, wherein the anxiety disorder is a phobia.

6. The method of claim 1, wherein the anxiety disorder is obsessive-compulsive disorder.

7. The method of claim 1, wherein the anxiety disorder is characterized by panic attacks.

8. The method of claim 1, wherein the protein synthesis inhibitor is a mTor protein synthesis inhibitor.

9. The method of claim 1, wherein the protein synthesis inhibitor is selected from the group consisting of rapamycin, CCI-779, and RAD-001.

10. The method of claim 1, wherein the eliciting event is encountering an environment which triggers a conditioned place preference in the subject.

11. The method of claim 1, wherein the eliciting event is a visual stimulus which triggers a conditioned place preference in the subject.

12. The method of claim 1, wherein the eliciting event is an olfactory stimulus which triggers a conditioned place preference in the subject.

13. The method of claim 1, wherein the eliciting event is an auditory stimulus which triggers a conditioned place preference in the subject.

14. The method of claim 1, wherein the eliciting event is a tactile stimulus which triggers a conditioned place preference in the subject.

15. The method of claim 1, wherein the eliciting event is a gustatory stimulus which triggers a conditioned place preference in the subject.

16. A method for inhibiting in a subject the onset of an anxiety disorder in response to an anxiety disorder-causing event, wherein the disorder would be characterized by symptoms which would be exacerbated by the subject's exposure to a predetermined eliciting event, which method comprises administering to the subject a prophylactically effective amount of a protein synthesis inhibitor before, during the time and/or after the subject intentionally recalls the eliciting event.

17. The method of claim 16, wherein the subject is human.

18. The method of claim 16, wherein the anxiety disorder is post-traumatic stress disorder caused by experiencing an act of violence.

19. The method of claim 16, wherein the protein synthesis inhibitor is a mTor protein synthesis inhibitor.

20. The method of claim 16, wherein the protein synthesis inhibitor is selected from the group consisting of rapamycin, CCI-779, and RAD-001.

21. An article of manufacture comprising (a) a packaging material having therein a protein synthesis inhibitor, and (b) a label indicating a use for the inhibitor in treating, or inhibiting the onset of, an anxiety disorder in a subject.
